(19) United States
(12) Patent Application Publication
(10) Pub. No.: US 2026/0315490 A1
(43) Pub. Date: Oct. 15, 2026

(54) PHARMACOLOGICAL TUBULAR ORGAN SMOOTH MUSCLE RELAXATION THROUGH RHO-ASSOCIATED PROTEIN KINASE INHIBITION

(71) Applicant: THE REGENTS OF THE UNIVERSITY OF CALIFORNIA, Oakland, CA (US)

(72) Inventors: Zachary E Tano, Orange, CA (US); Ralph V. Clayman, Orange, CA (US); Jaime Landman, Orange, CA (US); Andrei Dragos Cumpanas, Orange, CA (US); Yi Xi Wu, Orange, CA (US); Bruce Gao, Orange, CA (US); Sohrab Naushad Ali, Orange, CA (US); Roshan Patel, Orange, CA (US)

(21) Appl. No.: 19/645,070

(22) Filed: Apr. 10, 2026

Related U.S. Application Data
(60) Provisional application No. 63/786,939, filed on Apr. 10, 2025.

(51) Int. Cl. A61K 31/5513; A61K 31/4196; A61K 31/437; A61K 31/44; A61K 31/4439; A61K 31/472; A61K 31/4725; A61K 31/517; A61K 31/519

(52) U.S. Cl. CPC A61K 31/5513; A61K 31/4196; A61K 31/437; A61K 31/44; A61K 31/4439; A61K 31/472; A61K 31/4725; A61K 31/517; A61K 31/519

(57) ABSTRACT

Provided are methods of administering therapeutically effective dose(s) of administering therapeutically effective dose(s) of a Rho-associated protein kinase (ROCK) inhibitor to increase luminal distensibility of an organ in a subject, and applications thereof, including kidney stone removal. Also provided are methods of administering therapeutically effective dose(s) of a ROCK inhibitor to treat or facilitate treatment of kidney stones in a subject in need thereof.

### CROSS REFERENCE TO RELATED APPLICATIONS

This application claims priority under 35 U.S.C. § 119 from Provisional Application Ser. No. 63/786,939, filed Apr. 10, 2025 the disclosure of which is incorporated herein by reference.

### TECHNICAL FIELD

Provided here are methods of administering therapeutically effective dose(s) of a Rho-associated protein kinase (ROCK) inhibitor to increase luminal distensibility of an organ in a subject, and applications thereof, including kidney stone removal.

### BACKGROUND

Stone disease is considered benign, and treatment is indicated to relieve or prevent patient symptoms and other complications, such as sepsis resulting from a collecting system obstructed with infected urine or loss of renal function. Stone disease affects 1 in 10 Americans, and in 2014, there were 1 million to 2 million visits to the emergency department for renal colic. In addition to patient suffering, the cost to the U.S. taxpayer is projected to be $4.57 billion by 2030. Therefore, there is a great need for more effective treatment of renal colic and stone disease through prevention, medications, or surgical intervention.

### SUMMARY

Relaxing ureteral smooth muscle is an ongoing therapeutic area of interest for facilitating ureteroscopy, promoting spontaneous stone passage, and for possibly reducing renal colic. A ureteral force sensor was developed and used herein to both directly measure ureteral distensibility, and to allow safe passage of ureteral instruments at just under 8N in the human ureter. The studies presented herein evaluated the in vivo effect of six oral medications on porcine ureteral distensibility for the first time.

Thirty-nine juvenile female Yorkshire swine were orally administered either a placebo or one of six known smooth muscle relaxants for one week: fasudil, mirabegron, Rowatinex, silodosin, tadalafil, or verapamil. Ureteral distensibility was assessed using a ureteral force sensor disclosed herein by the sequential passage of urethral dilators, in 2 Fr increments, prior to and after seven days of drug administration. Blood was obtained on day zero and day seven, to quantify serum medication levels. Urine cultures were obtained at day zero and day seven. A generalized linear model, adjusted for confounders such as age and bacteriuria, was developed to evaluate the effect of each medication on ureteral distensibility, based on the maximum size dilator passed and a more detailed ureteral size scale (USS) compared to the control group.

It was found in the studies presented herein that significant increases in the maximum size dilator passed were observed for animals given fasudil (1.216 Fr or 0.41 mm, p=0.026) and verapamil (0.057 Fr or 0.02 mm, p=0.002). Only animals given fasudil, however, significantly increased their ureteral size scale: 3.685 (equivalent to 1.228 Fr, or 0.41 mm, p=0.028). Accordingly, fasudil, a rho-kinase inhibitor, had the greatest impact, increasing ureteral distensibility by 1.216 Fr (0.41 mm). A size increase of this magnitude has implications for passing larger instruments through the ureter safely, medical expulsive therapy for passing ureteral stones, and reducing ureteral stent colic.

Although the studies presented herein were in ureters, increased luminal distensibility can be applied to all organs in which a lumen is surrounded by smooth muscle, including the prostate, bladder, penis, and gastrointestinal organs such as the gallbladder, gallbladder duct, and other organs, such as bronchioles in the lungs.

In a particular embodiment, the disclosure provides a method to increase luminal distensibility of an organ in a subject, comprising: administering a therapeutically effective dose of a Rho-associated protein kinase (ROCK) inhibitor to the subject, wherein administration of the therapeutically effective dose of the ROCK inhibitor results in increased luminal distensibility in the organ of the subject. In another embodiment, or further embodiment of any of the foregoing embodiments, the organ comprises a lumen surrounded by smooth muscle. In another embodiment, or further embodiment of any of the foregoing embodiments, the organ is selected from ureter(s), prostate, bladder, penis, gallbladder, gallbladder duct, and lung bronchiole. In another embodiment, or further embodiment of any of the foregoing embodiments, the organ is ureter(s) or prostate. In another embodiment, or further embodiment of any of the foregoing embodiments, the ROCK inhibitor is selected from fasudil, hydroxyfasudil, belumosudil, ripasudil, netarsudil, zelasudil, ROC-101, OXA-06, ROCK inhibitor-2, H-1152P, CPL409116, AT3148, RKI-1447, FSD-C10, verosudil, sovesudil, cotosudil, and a derivative or analog of any of the foregoing. In another embodiment, or further embodiment of any of the foregoing embodiments, the therapeutically effective dose of the ROCK inhibitor is administered orally or intravenously to the subject. In another embodiment, or further embodiment of any of the foregoing embodiments, the ROCK inhibitor is fasudil, hydroxyfasudil, or a fasudil derivative or analog. In another embodiment, or further embodiment of any of the foregoing embodiments, the fasudil derivative or analog is selected from ripasudil, H-1152, dimethylfasudil and Rho Kinase Inhibitor IV. In another embodiment, or further embodiment of any of the foregoing embodiments, fasudil, hydroxyfasudil or a fasudil derivative or analog is administered at a dose of 10 mg to 100 mg. In another embodiment, or further embodiment of any of the foregoing embodiments, the subject is administered daily from 1 to 4 doses of fasudil, hydroxyfasudil and/or a fasudil derivative or analog. In another embodiment, or further embodiment of any of the foregoing embodiments, the subject has a condition or disorder that can be treated by increasing luminal distensibility of the organ. In another embodiment, or further embodiment of any of the foregoing embodiments, the subject has kidney stone(s). In another embodiment, or further embodiment of any of the foregoing embodiments, the subject is a human patient.

In a particular embodiment, the disclosure also provides a method to increase luminal distensibility of an organ in a subject, comprising: administering a therapeutically effective dose of fasudil, a hydroxyfasudil and/or a fasudil analog or derivative to the subject, wherein administration of the therapeutically effective dose of fasudil, a hydroxyfasudil and/or a fasudil analog or derivative results in increased luminal distensibility in the organ of the subject. Examples of fasudil derivatives or analogs include, but are not limited to, ripasudil, H-1152, dimethylfasudil and Rho Kinase Inhibitor IV. In another embodiment, or further embodiment of any of the foregoing embodiments, the organ comprises a lumen surrounded by smooth muscle. In another embodiment, or further embodiment of any of the foregoing embodiments, the organ is selected from ureter(s), prostate, bladder, penis, gallbladder, gallbladder duct, and lung bronchiole. In another embodiment, or further embodiment of any of the foregoing embodiments, the organ is selected from ureter(s) and prostate. In another embodiment, or further embodiment of any of the foregoing embodiments, the therapeutically effective dose of fasudil, a hydroxyfasudil and/or a fasudil analog or derivative is administered orally or intravenously to the subject. In another embodiment, or further embodiment of any of the foregoing embodiments, fasudil, a hydroxyfasudil and/or a fasudil analog or derivative is administered at a dose of 10 mg to 100 mg. In another embodiment, or further embodiment of any of the foregoing embodiments, the subject is administered daily from 1 to 4 doses of fasudil, a hydroxyfasudil and/or a fasudil analog. In another embodiment, or further embodiment of any of the foregoing embodiments, the subject has a condition or disorder that can be treated by increasing luminal distensibility of the organ. In another embodiment, or further embodiment of any of the foregoing embodiments, the subject has kidney stone(s). In another embodiment, or further embodiment of any of the foregoing embodiments, the subject is a mammalian subject. In yet another embodiment, the mammalian subject is a human subject.

In a particular embodiment, the disclosure further provides a method to treat or facilitate treatment of kidney stone(s) in a subject in need thereof, comprising: administering a therapeutically effective dose of a Rho-associated protein kinase (ROCK) inhibitor to the subject, wherein administration of the therapeutically effective dose of the ROCK inhibitor results in increased ureteral distensibility. In another embodiment, or further embodiment of any of the foregoing embodiments, administration of the therapeutically effective dose of a ROCK inhibitor to the subject in need thereof, facilitates kidney stone removal by a medical procedure or by spontaneous passing. In another embodiment, or further embodiment of any of the foregoing embodiments, wherein the medical procedure is ureteroscopy. In another embodiment, or further embodiment of any of the foregoing embodiments, wherein administration of the therapeutically effective amounts of the ROCK inhibitor allows for usage of larger diameter medical instruments that were not safely usable prior to administration. In another embodiment, or further embodiment of any of the foregoing embodiments, wherein the ROCK inhibitor is selected from fasudil, hydroxyfasudil, belumosudil, ripasudil, netarsudil, zelasudil, ROC-101, OXA-06, ROCK inhibitor-2, H-1152P, CPL409116, AT3148, RKI-1447, FSD-C10, verosudil, sovesudil, cotosudil, and a derivative or analog of any of the foregoing. In another embodiment, or further embodiment of any of the foregoing embodiments, wherein the therapeutically effective dose of the ROCK inhibitor is administered orally or intravenously to the subject. In another embodiment, or further embodiment of any of the foregoing embodiments, wherein the ROCK inhibitor is fasudil, hydroxyfasudil, or a fasudil derivative or analog. Examples of fasudil derivatives or analogs include, but are not limited to, ripasudil, H-1152, dimethylfasudil and Rho Kinase Inhibitor IV. In another embodiment, or further embodiment of any of the foregoing embodiments, the fasudil derivative or analog is selected from ripasudil, H-1152, dimethylfasudil and Rho Kinase Inhibitor IV. In another embodiment, or further embodiment of any of the foregoing embodiments, fasudil, hydroxyfasudil or a fasudil derivative or analog is administered at a dose of 10 mg to 100 mg. In another embodiment, or further embodiment of any of the foregoing embodiments, the subject is administered daily from 1 to 4 doses of fasudil, hydroxyfasudil and/or a fasudil derivative or analog. In another embodiment, or further embodiment of any of the foregoing embodiments, the subject is a human patient.

In a particular embodiment, the disclosure also provides a method to treat or facilitate treatment of kidney stones in a subject in need thereof, comprising: administering a therapeutically effective dose of fasudil, a hydroxyfasudil and/or a fasudil analog or derivative, to the subject in need thereof, wherein administration of the therapeutically effective dose of fasudil, a hydroxyfasudil and/or a fasudil analog or derivative results in increased ureteral distensibility. Examples of fasudil derivatives or analogs include, but are not limited to, ripasudil, H-1152, dimethylfasudil and Rho Kinase Inhibitor IV. In another embodiment, or further embodiment of any of the foregoing embodiments, administration of the therapeutically effective dose of fasudil, a hydroxyfasudil and/or a fasudil analog or derivative, to the subject in need thereof, facilitates kidney stone removal by a medical procedure or by spontaneous passing. In another embodiment, or further embodiment of any of the foregoing embodiments, the medical procedure is ureteroscopy. In another embodiment, or further embodiment of any of the foregoing embodiments, administration of the therapeutically effective amounts of fasudil, a hydroxyfasudil and/or a fasudil analog or derivative allows for usage of larger diameter medical instruments that were not safely usable prior to administration. In another embodiment, or further embodiment of any of the foregoing embodiments, the therapeutically effective dose of fasudil, a hydroxyfasudil and/or a fasudil analog or derivative is administered orally or intravenously to the subject. In another embodiment, or further embodiment of any of the foregoing embodiments, fasudil, a hydroxyfasudil and/or a fasudil analog or derivative is administered at a dose of 10 mg to 100 mg. In another embodiment, or further embodiment of any of the foregoing embodiments, the subject is administered daily from 1 to 4 doses of fasudil, a hydroxyfasudil and/or a fasudil analog. In another embodiment, or further embodiment of any of the foregoing embodiments, the subject is a mammalian subject. In another embodiment, or further embodiment of any of the foregoing embodiments, the mammalian subject is a human subject.

In a certain embodiment, the disclosure provides for a composition, method or kit as substantially described in the specification and/or drawings.

### DESCRIPTION OF DRAWINGS

The accompanying drawings, which are incorporated into and constitute a part of this specification, illustrate one or more embodiments of the disclosure and, together with the detailed description, explain the invention's principles and implementations.

FIG. 1 presents an embodiment of a diagram of the study design for testing oral smooth muscle relaxants (SMRs) effect on ureteral distensibility. Part 1: Factors leading to larger ureters were determined by pooling together female Yorkshire swine whose ureters were measured with the same methods prior to any experimental intervention to determine both the average ureteral size and factors leading to larger ureters through a binary logistic regression. Part 2: Ureteral size differences between pre- and post-treatment of each medication were compared to the control group with a generalized linear model that was adjusted for the factors leading to more distensible ureters.

FIG. 2 presents inclusion and exclusion criteria for the studies presented herein. Ureters were evaluated as a single unit and excluded from the study if a PULS grade ≥3 injury occurred or a 10 Fr urethral dilator failed to pass (technical error) at 3.5 N. The ureters included for each group were as follows: fasudil (n=10), mirabegron (n=9), Rowatinex® (n=4), tadalafil (n=10), verapamil (n=4), and control (n=10).

FIG. 3 provides a schematic of ureteral anatomy and shows the location of maximum urethral dilator failure to pass is based on three ureteral sites (i.e., distal, mid, and proximal). The location of the maximum urethral dilator failure to pass was defined using three anatomical landmarks. 1) distal ureter—between the ureteral orifice and the pelvic brim, 2) mid-ureter—between the pelvic brim and half the distance between the pelvic brim and the ureteropelvic junction (UPJ), and 3) proximal ureter—between half the distance between the pelvic brim and UPJ to the UPJ.

FIG. 4 demonstrates intra-animal variability in ureteral size based on ureteral size scale (USS). Histogram showing the distribution of absolute intra-animal differences in dilation score using the Ureteral Size Scale (USS). Ureters were paired within each animal without regard to anatomical laterality. While 68.14% of ureter pairs differed by ≤3 USS points (35.40% with no difference, USS=0; 32.74% had a difference of 1-3 points), 31.86% exhibited a difference ≥3 points, reflecting notable variability in baseline size between ureters within the same animal. The analysis was conducted using a pooled cohort of 113 female juvenile Yorkshire swine from prior studies where both ureters were sized prior to any experimental intervention.

FIG. 5 presents serum drug concentration based on mass spectrometry analysis. No medication was detected in any group on day zero. On day seven, the mean serum concentrations (ng/mL) in each group were 9.26 for hydroxyfasudil, 0.83 for mirabegron, 263.01 for silodosin, 52.26 for tadalafil, and 26.65 for verapamil. The estimated human serum concentrations (ng/mL) are shown as dashed lines: 12 for hydroxyfasudil, 30 for mirabegron, 51 for silodosin, 450 for tadalafil, and 40 for verapamil. Note that Rowatinex® was not analyzed.

FIG. 6 shows control group ureteral size at baseline compared to day seven. There was no significant difference between the mean ureteral sizes of the control group in diameter (Fr) or USS.

### DETAILED DESCRIPTION

As used herein and in the appended claims, the singular forms “a”, “an”, and “the” include plural referents unless the context clearly dictates otherwise. Thus, for example, reference to “a smooth muscle” includes a plurality of such smooth muscles and reference to “the ROCK inhibitor” includes reference to one or more ROCK inhibitors and equivalents thereof known to those skilled in the art, and so forth.

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

Also, the use of “or” means “and/or” unless stated otherwise. Similarly, “comprise,” “comprises,” “comprising,” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “pharmaceutically acceptable” as used herein, refers to a pharmaceutically acceptable material, composition, or vehicle, such as a liquid or solid filler, diluent, excipient, solvent, or encapsulating material. Each component should be “pharmaceutically acceptable” in the sense of being compatible with the other ingredients of a pharmaceutical formulation. It should also be suitable for use in contact with the tissue or organ of humans and animals without excessive toxicity, irritation, allergic response, immunogenicity, or other problems or complications, commensurate with a reasonable benefit/risk ratio. Examples of “pharmaceutically acceptable carriers” and “pharmaceutically acceptable excipients” can be found in the following, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004.

The term “subject” as used herein, refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein. For example, a mammalian subject can refer to a human patient.

The term “therapeutically effective amount” refers to the amount of a compound(s) that, when administered, is sufficient to prevent development of, or alleviate to some extent, one or more of the symptoms of the disease, disorder or condition being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The terms “treat,” “treating,” and “treatment,” as used herein, refers to ameliorating symptoms associated with a disease, disorder, or condition (e.g., a neurodegenerative disease), including preventing or delaying the onset of the disease or disorder symptoms, and/or lessening the severity or frequency of symptoms of the disease or disorder.

The term “fasudil analog or derivative” refers to a compound or substance that is structurally related to fasudil by retaining the isoquinoline sulfonamide structure of fasudil to inhibit ROCK1 and ROCK2 activity. Specific examples of fasudil analogs or derivatives include, but are not limited to, ripasudil, H-1152P, dimethylfasudil and Rho Kinase Inhibitor IV.

All publications mentioned herein are incorporated by reference in full for the purpose of describing and disclosing methodologies that might be used in connection with the description herein. The publications are provided solely for their disclosure prior to the filing date of the present application. Nothing herein is to be construed as an admission that the inventors are not entitled to antedate such disclosure by virtue of prior disclosure. Moreover, with respect to any term that is presented in one or more publications that is similar to, or identical with, a term that has been expressly defined in this disclosure, the definition of the term as expressly provided in this disclosure will control in all respects.

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated by reference in full for the purpose of describing and disclosing methodologies that might be used in connection with the description herein. The publications are provided solely for their disclosure prior to the filing date of the present application. Nothing herein is to be construed as an admission that the inventors are not entitled to antedate such disclosure by virtue of prior disclosure. Moreover, with respect to any term that is presented in one or more publications that is similar to, or identical with, a term that has been expressly defined in this disclosure, the definition of the term as expressly provided in this disclosure will control in all respects.

Stone disease is prevalent, expensive, and morbid. Recurrences following ureteroscopic lithotripsy are related to residual fragments not removed at the time of the procedure. One way to mitigate this situation is to pass a larger, 16 Fr, access sheath or to consider aspiration of fragments exemplified by the 11.9 Fr, 2.50 mm aspiration channel Calyxo device via a 14 Fr access sheath. Unfortunately, even a 14 Fr access sheath resulted in significant ureteral injury in up to 26% of patients. To date, no smooth muscle relaxant (SMR) has been purposed for facilitating passage of larger ureteral access sheaths. Similarly, current SRMs aimed at successful medical expulsive therapy (MET) or stent colic relief remain controversial due to conflicting efficacy reports or studies limited only to distal ureteral calculi.

Surgical interventions aim to completely remove stones from the renal collecting system because any remaining stone fragment has the potential to be problematic for the patient, which leads to retreatment rates as high as 23% for ureteroscopic stone removal (URS) and 35% for percutaneous nephrolithotomy (PCNL) within one year. Besides painful recurrences for the patient, retreatment for stone disease more than doubles the cost for both procedure types. PCNL is the most effective intervention for removing a large stone burden in a single operative procedure. This approach, however, is associated with a higher rate of high-grade complications compared to ureteroscopy (URS) or extracorporeal shockwave lithotripsy (ESWL). While URS is more effective than ESWL for small stones, it remains less effective than PCNL for large stones (i.e., >1.5 cm). Emerging technologies, such as ureteroscopic aspiration functionality enabled by Thulium laser lithotripsy that breaks the stone apart into dust-like fragments compared to Holmium lasers, hold promise for improving the efficacy of ureteroscopy. Although not yet formally studied, the efficiency of aspiration URS is believed to be influenced by the diameter of the aspiration channel.

In addition to the stone treatments described above, temporary surgical interventions, such as ureteral stent placement, are also common. A ureteral stent allows urine to pass freely from the kidney to the bladder, preventing ureteral obstruction. Ureteral stents are utilized in a variety of situations, including (1) bypassing an obstructing ureteral stone, (2) preventing obstruction due to inflammation after instrumentation of the ureter, (3) promoting proper healing after iatrogenic ureteral injuries or surgical incisions of the ureter, and (4) chronic management of strictures or ureteral dysfunction. Stents are very useful but are not without complications. Indeed, after stent placement, patients experience various side effects. In one study, the common side effects of discomfort or urinary frequency with urgency occurred in up to 45% and 55% of stented patients, respectively. Unfortunately, there are currently no predictors to specifically identify which patients will experience these side effects.

Other than placing a stent several days prior to ureteral instrumentation, there are currently no other procedures that reliably increase the distensibility of the ureter. Medications have been explored for this purpose. The findings, however, remain controversial. Relaxing ureteral smooth muscle has been investigated because it is central to both medical expulsive therapy (MET), a treatment used to facilitate passage of ureteral stones and for renal colic relief due to obstructive ureteral stones or stents. Of note, the same medications used for MET have been employed to reduce colic and lower urinary tract symptoms (LUTS) following stent placement. Alpha-adrenergic antagonists, beta-adrenergic agonists, calcium channel antagonists, phosphodiesterase five (PDE5) inhibitors, and terpenes have all been explored for MET to treat obstructive ureteral stones.

Alpha-adrenergic antagonists are the most common class of medications prescribed for MET. Their mechanism of action (MOA) relies on the competitive binding of alpha-1-adrenergic receptors throughout the ureter and prevents norepinephrine binding. Molecular biology studies have shown that the alpha-1 receptors have a higher receptor density in the distal ureter than in the proximal and mid ureter. Furthermore, the alpha 1D receptor subtype is expressed more than the alpha 1A receptor subtype. Indeed, clinical controversy exists. When contrasting two multi-institution, double-blinded, randomized, placebo-controlled clinical studies, one study found an increased distal stone passage rate (4-10 mm) utilizing silodosin, which antagonizes the 1A receptor subtype. There was no difference in stone passage rate when considering all levels of the ureter. In contrast, a second study found that there was no difference in distal stone passage rate when considering all stone sizes (≤10 mm), but there was an increase in distal stone passage in the larger stone subgroup (5-10 mm) utilizing tamsulosin, which acts on the 1A and 1D receptor subtypes (number of patients with successful stone passage prescribed tamsulosin vs. placebo: 30 vs. 25 (22%); p=0.03, CI 3%-42%).

Mirabegron is the most common beta-adrenergic agonist utilized for MET. The mechanism of action for mirabegron relies on binding to the beta-3 receptor subtype, resulting in the relaxation of smooth muscles. Beta-adrenergic receptors are found in the human ureter, and human ex vivo experiments showed that antagonizing the beta 2 or 3 receptor subtypes induced ureteral relaxation. Just as with alpha-adrenergic antagonists, clinical controversy exists over the use of mirabegron for ureteral relaxation. A meta-analysis of randomized, controlled studies suggested that mirabegron might be effective for distal ureteral stone MET, and the benefit was shown in both small (<6 mm) and distal stones. The authors note, however, that there was heterogeneity among the studies. In general, autonomic nerves in the human ureter are more densely distributed in the distal portion, which might explain the possible MET benefits of adrenergic modulating medications acting distally.

Medications such as verapamil and nifedipine antagonize voltage-gated calcium channels: K channel and L-type. Both K and L-type calcium channels are present in human ureteral smooth muscle, and interference with these channels disrupts human ureteral force and frequency of contractions in ex vivo studies. In one multicenter, randomized, placebo-controlled clinical study, neither nifedipine nor tamsulosin decreased the need for further treatment of symptomatic ureteral stones (<5 mm and >5 mm, all three levels of the ureter considered) compared to placebo in patients deemed appropriate for MET. Conversely, a second randomized clinical study found that nifedipine plus a steroid increased stone expulsion rate (distal stones <10 mm) compared to control.

PDE5 inhibitors have been extensively studied in the context of erectile dysfunction (ED). Nitric oxide (NO) is a neurotransmitter produced by the conversion of L-arginine to L-citrulline via nitric oxide synthase (NOS) following sexual stimulation, and NO activates guanyl cyclase (GC) to produce cyclic guanosine monophosphate (cGMP), which acts as a secondary messenger to regulate calcium channels that relax smooth muscle. PDE is an enzyme that eliminates cGMP, and PDE5 inhibitors prevent the breakdown of cGMP. A dual PDE5/PDE1 inhibitor was the first example of a PDE inhibitor found to reduce ex vivo human ureter tension, which was followed by specific PDE5 inhibitors, including tadalafil. One prospective, randomized, controlled trial showed that for patients with distal ureteral stones (5-10 mm), tadalafil had a higher expulsion rate compared to tamsulosin. A second prospective, randomized, placebo-controlled study found that patients with distal ureteral stones (4-10 mm) prescribed tadalafil had a higher rate of stone expulsion than those prescribed placebo but no different than those prescribed tamsulosin. Of note, not all studies found benefits. Indeed, in another prospective, randomized, double-blinded clinical trial, there was no difference in stone expulsion for patients with distal ureteral stones (<10 mm) in the tadalafil, tamsulosin, or placebo groups.

Terpenes are a heterogenous group of oils derived from hydrocarbons; for example, Rowatinex®, is a medication composed of a mixture of terpene compounds (31% pinene, 15% camphene, 10% borneol, 4% anethole, 4% fenchone, 3% cineole). Terpenes have demonstrated spasmolytic activity in smooth muscle organs in animal studies. Two randomized controlled trials that utilized Rowatinex® in MET and favored Rowatinex® compared to placebo. Conversely, other investigators performed a prospective, randomized, controlled trial comparing Rowatinex® to tamsulosin and diclofenac, and only tamsulosin had an increased stone expulsion rate compared to the diclofenac group.

Overall, there is no clear consensus on the most effective medication for MET. Clearly, the development of an alternative medication that is effective along the entire ureter would be a valuable addition to the current MET options. Moreover, if this medication were truly effective in causing ureteral smooth muscle relaxation, it would also greatly facilitate ureteroscopic stone removal procedures.

For renal/stent colic, the same ureteral relaxation principle behind MET is employed when considering pain relieving medication outside of the non-steroidal anti-inflammatory drug or opioid medication classes. Animal studies with a subjective parameter of discomfort that often coincides with the non-subjective parameter of increased voiding frequency either cannot be reliably measured or are inhumane to study in an awake animal respectively; therefore, these in vivo studies are not possible, and clinical information has been the only way to assess the efficacy of these potentially smooth muscle relaxation medications.

The alpha-adrenergic antagonists, tamsulosin and silodosin, have been reported to have no difference in patient emergency department visits, hospital admissions, or use of analgesics in one study and no difference in patient time to passage, pain episodes, or analgesic requirements in a second study. The beta-adrenergic agonist mirabegron had no difference in patient pain episodes. The calcium channel antagonist nifedipine was found to have no difference in patient analgesia requirements or time to passage in one study. Patients in a second study, however, did have reduced diclofenac requirements with the caveat that this study employed steroids with nifedipine, which also have anti-inflammatory properties. The PDE5 inhibitor tadalafil did have a lower patient analgesic requirement, less time to stone expulsion, and fewer hospital visits. Finally, patients prescribed Rowatinex® experienced no difference in colic episodes or analgesic use. Overall, tadalafil had the overall best evidence of treating renal/stent colic.

Fasudil, and its active metabolite, hydroxyfasudil, is a non-specific ROCKi that competes with ATP to inhibit both rho-kinase isoforms Rho-kinase β3/ROK/β4/ROCK1 (ROCK1) and Rho-kinase α3/ROKα4/ROCK2 (ROCK2). The most studied Rho-kinase (ROCK) mechanism of action involves the myosin light chain complex. Rho-kinase inhibitors (ROCKi) attenuate the inhibition of the myosin light chain phosphatase (MLCP), which promotes smooth muscle relaxation through dephosphorylation of the myosin light chain complex. Although the two ROCK isoforms appear to have different roles in contractility, the functional differences remain unclear. Moreover, ROCK has been shown to increase the intensity of muscle contraction provided the Ca²⁺ levels remain constant, termed Ca²⁺ sensitivity. ROCKi's ability to induce ureteral relaxation is therefore not surprising. Furthermore, ROCK2 has been found to shift T cells and macrophages from a pro-inflammatory to an anti-inflammatory phenotype. ROCKi has mainly been studied in a cardiovascular context, in which its benefits have been attributed to smooth muscle relaxation, an increase in nitric oxide production, and the promotion of anti-inflammatory immune cell functions. Accordingly, in addition to potentially facilitating the passage of larger instruments through its smooth muscle relaxation effect, the combination of smooth muscle relaxation and a reduced inflammatory response could further increase the likelihood of spontaneous passage of a ureteral calculus and possibly reduce stent colic. Although ROCK's presence has been demonstrated in the human ureter smooth muscle and ROCKi Y-27632 has been shown to attenuate human ureter smooth muscle tension and contractile responses, to date, ROCKi's potential urological implications have largely gone untested. A previous study using Y-27632, a non-selective ROCKi, showed that Y-27632 in combination with nifedipine may have a beneficial effect on ureteral contraction frequency and amplitude. Y-27632, however, is very non-selective ROCKi, and the beneficial effects were only realized when Y-27632 was administered in a combined therapy with nifedipine. Lacking from these studies is any direct measurement of actual ureteral distensibility, which as tested in the current study is the most clinically relevant objective measurement.

A medication that acutely increases ureteral distensibility by relaxing smooth muscle combined with the use of a force sensor will increase the ureteral diameter and permit safe passage of larger access sheaths and ureteroscopes. Larger instruments contain larger aspiration channels and subsequently might augment the efficacy of ureteroscopic stone removal while allowing it to be applied to even larger stones. Initial studies with electromotive drug administration therapy demonstrated marginal efficacy with administration being over a clinically unfeasible 20-minutes. Prior to the results presented herein, no studies to date have shown a direct clinical effect of any medication on ureteral distensibility. Accordingly, there is a need for a medication that acts along the entire ureter.

The average size of the human ureter is calculated to be 15 Fr and that ureteral injuries can be prevented by inserting instruments under a 6 Newton (N) force threshold. Without a force sensor, 46% of urologists using a 14 Fr ureteral access sheath were found to cause ureteral injuries. These ureteral size parameters and safety data highlight that larger ureteral instruments and aspiration tools can be safely passed with the integration of a force sensor into clinical practices.

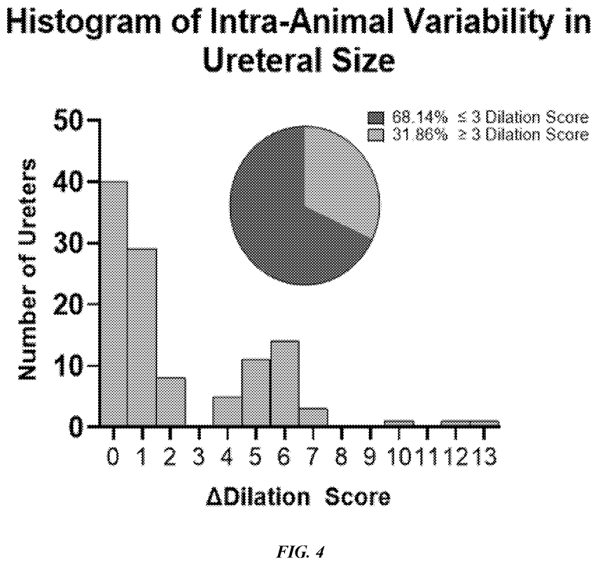

There is precedence but not consensus on how to experimentally evaluate the ureter. In our human studies, there were few bilateral procedures, and thus, in those cases, ureters were considered individually. Clinical asymmetry in pathological renal processes is well documented such as stone formation, urothelial carcinoma, and pyelonephritis. As such, each of the swine ureters were considered in the study as an independent experimental unit, rather than biological replicates. Indeed, the experimental observations also supported differences between the ipsilateral and contralateral ureter in the same animal in terms of baseline ureteral distensibility (see FIG. 4).

To date, there has not been a functional study of a broad range of oral SRMs regarding their ability to increase ureteral distensibility. Herein, is presented the first in vivo study of six oral SRMs (fasudil (rho-associated protein kinase (ROCK) inhibitor), mirabegron (beta-3-adrenergic receptor agonist), Rowatinex®, (terpene mixture), silodosin (non-specific alpha-adrenergic antagonist), tadalafil (phosphodiesterase five inhibitor), and verapamil (calcium channel blocker)) with respect to their ability to augment porcine ureteral distensibility, using a physiological safe force threshold of 3.5N.

In the studies presented herein, fasudil and hydroxyfasudil increased ureteral distensibility whereas alpha-adrenergic antagonists, beta-adrenergic agonists, PDE5 inhibitors, and terpenes did not have an effect on ureteral distensibility. Verapamil, a calcium channel blocker, resulted in ureteral smooth muscles relaxation, albeit to a much lesser extent. Fasudil and hydroxyfasudil have the added benefit of not acting upon extracellular receptors and, therefore, address the ureteral smooth muscle non-specifically while maintaining a safe and tolerable side effect profile as described in several clinical trials for non-urologic applications.

Except for fasudil and hydroxyfasudil, the various oral medications used in the studies presented herein had previously, in the laboratory or clinical setting, been shown to be effective either with regard to MET, renal colic relief or both. The lack of clinically relevant distensibility for verapamil had less of a precedent given that only ex vivo studies specific for verapamil exist, and clinical studies results for nifedipine regarding MET have been conflicting.

Among the six drugs tested, fasudil and hydroxyfasudil had a significant impact on increasing ureteral distensibility. The studies provided herein give direct evidence of ROCK presence in the human ureter and corroborate the biological function of ROCKi's. The unique methodologies of the disclosure atraumatically and directly quantified changes in ureteral diameter. As such, in vivo measurements of ureteral distensibility can be directly quantified, which prior to, could only be inferred using existing methods. Indeed, in the studies presented herein, after adjusting for age and bacteriuria, fasudil was the only medication that significantly increased ureteral distensibility: 1.216 Fr (+0.41 mm) and USS (+3.685).

Fasudil has been used for the treatment of cerebral vasospasm, which is often due to subarachnoid hemorrhage, as well as to improve the cognitive decline seen in stroke patients. It has been found to be effective for the treatment of pulmonary hypertension. It has been demonstrated that fasudil could improve memory in normal mice, identifying the drug as a possible treatment for age-related or neurodegenerative memory loss. It has been approved for use in Japan and China since 1995.

It was found in the studies presented herein, that significant increases in the maximum size dilator passed were observed for animals given fasudil (1.216 Fr, p=0.026) and verapamil (0.057 Fr, p=0.002). Only animals given fasudil, however, significantly increased their ureteral size scale: 3.685 (equivalent to 1.228 Fr, p=0.028). Accordingly, fasudil, a rho-kinase inhibitor, had the greatest impact, increasing ureteral distensibility by 1.216 Fr (0.44 mm). The results of the studies presented herein provide a potential, novel pharmacologic adjunct for MET of ureteral urolithiasis given that even a small increase in ureteral distensibility could have a favorable clinical impact on passage of a ureteral stone. This is in concert with other data in which some preserved peristalsis, as noted after local ROCKi treatment, could prove helpful in advancing a urolith further distally toward the bladder. There are also renal colic relief possibilities given the SMR mechanism of action of fasudil which might allow urine to more easily flow around the stone due to augmented ureteral distensibility. In addition, fasudil has demonstrated safety in clinical evaluations among patients with angina, at doses far exceeding those used in the present study. Studies to optimize the medication delivery method and timing, taking into account fasudil pharmacokinetics, would be beneficial with regard to the passage of larger ureteral access sheaths thereby facilitating the ureteroscopic treatment of renal calculi larger than 2 cm.

Although the studies presented herein were in the ureter, increased luminal distensibility can be applied to all organs in which a lumen is surrounded by smooth muscle, including the prostate, bladder, penis, and gastrointestinal organs such as the gallbladder, gallbladder duct, and other organs, such as bronchioles in the lungs. Currently, treatment strategies revolve around relaxing the smooth muscle around the prostate to control symptoms using alpha-1-adrenergic antagonists, PDE5 inhibitors, and 5ARI that have efficacy alone or in combination. Unfortunately, complete symptom relief is not always accomplished. BPH is multifactorial, and 5ARI address only one of the multiple BPH etiologies, but 5ARI have reduced efficacy in inflammatory environments, which is one of the BPH etiologies. ROCKi has the potential to address many of the multifactorial causes of BPH, including the focus of the findings on smooth muscle relaxation with fasudil and hydroxyfasudil as well as having an anti-inflammatory component. No other medication currently in use addresses the multiple etiologies of BPH in addition to symptom relief, which is why fasudil could be a powerful addition to treat BPH.

A major strength of the studies presented herein is the use of a unique, ureteral-specific force-sensor. This force sensor has been used to accurately and atraumatically assess the distensibility of the swine ureter as well as the human ureter intraoperatively. This is a departure from other studies in which only indirect measurement methods were used to imply ureteral smooth muscle relaxation, such as peristalsis frequency and intraluminal pressure changes.

Another strength of the studies presented herein is the assessment of a control group at time zero and seven days, confirming that the ureteral measurement methods were not associated with a change in the size of the ureter between the first and second measurement in the same animal nor did the one-week interval account for any appreciable increase in ureteral size among the control animals.

A pharmaceutical composition comprising a ROCK inhibitor disclosed herein (e.g., fasudil, hydroxyfasudil, etc.) can be in a form suitable for administration to a subject using carriers, excipients, and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, buffered saline, alcohols, glycerol, and polyhydric alcohols. Intravenous vehicles include fluid and nutrient replenishers. Preservatives include antimicrobial, chelating agents, and inert gases. Other pharmaceutically acceptable carriers include aqueous solutions, non-toxic excipients, including salts, preservatives, buffers and the like, as described, for instance, in Remington's Pharmaceutical Sciences, 15th ed., Easton: Mack Publishing Co., 1405-1412, 1461-1487 (1975), and The National Formulary XIV., 14th ed., Washington: American Pharmaceutical Association (1975), the contents of which are hereby incorporated by reference. The pH and exact concentration of the various components of the pharmaceutical composition are adjusted according to routine skills in the art. See Goodman and Gilman's, The Pharmacological Basis for Therapeutics (7th ed.).

The disclosure further provides for a pharmaceutical composition comprising a ROCK inhibitor disclosed herein (e.g., fasudil, hydroxyfasudil, etc.) is administered by injection (subcutaneous, intravenous, etc.), oral administration, inhalation, transdermal application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. In all cases, the composition should be sterile and should be fluid to the extent that easy syringability exists. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be typical to include isotonic agents, for example, sugars, polyalcohols, such as mannitol, sorbitol, or sodium chloride in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

The pharmaceutical composition comprising a ROCK inhibitor disclosed herein (e.g., fasudil, hydroxyfasudil, etc.) can be orally administered, for example, with an inert diluent or an assimilable edible carrier. The pharmaceutical composition and other ingredients can also be enclosed in a hard or soft-shell gelatin capsule, compressed into tablets, or incorporated directly into the individual's diet. For oral therapeutic administration, the pharmaceutical composition can be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations can, of course, be varied and can conveniently be between about 5% to about 80% of the weight of the unit.

The tablets, troches, pills, capsules, and the like can also contain the following: a binder, such as gum tragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Of course, any material used in preparing any dosage unit form should be pharmaceutically pure and substantially non-toxic/biocompatible in the amounts employed. In addition, the pharmaceutical composition can be incorporated into sustained-release preparations and formulations.

Thus, a “pharmaceutically acceptable carrier” is intended to include solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the pharmaceutical composition, use thereof in the therapeutic compositions and methods of treatment is contemplated. Supplementary active compounds can also be incorporated into the compositions.

It is especially advantageous to formulate parenteral compositions in dosage unit form for ease of administration and uniformity of dosage. “Dosage unit form” as used herein, refers to physically discrete units suited as unitary dosages for the individual to be treated; each unit containing a predetermined quantity of pharmaceutical composition is calculated to produce the desired therapeutic effect in association with the required pharmaceutical carrier. The specifics for the dosage unit forms of the disclosure are related to the characteristics of the pharmaceutical composition and the particular therapeutic effect to be achieve.

The principal pharmaceutical composition is compounded for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

In a particular embodiment, the disclosure provides a method to increase luminal distensibility of an organ in a subject, comprising: administering a therapeutically effective dose of a ROCK inhibitor disclosed herein (e.g., fasudil, hydroxyfasudil, etc.) to the subject. In a further embodiment, administration of the therapeutically effective amounts of a ROCK inhibitor disclosed herein (e.g., fasudil, hydroxyfasudil, etc.) results in increased luminal distensibility in the organ of the subject. In another embodiment, the organ comprises a lumen surrounded by smooth muscle. Examples of such organs include, but are not limited to, ureter(s), prostate, bladder, penis, gallbladder, gallbladder duct, and lung bronchiole, and the like. In a further embodiment, the organ is selected from ureter(s) and prostate. Examples of ROCK inhibitors include, but are not limited to, fasudil, hydroxyfasudil, belumosudil, ripasudil, netarsudil, zelasudil, ROC-101, OXA-06, ROCK inhibitor-2, H-1152P, CPL409116, AT3148, RKI-1447, FSD-C10, verosudil, sovesudil, cotosudil, and derivatives or analogs of any of the foregoing. In a certain embodiment, the therapeutically effective dose of a ROCK inhibitor disclosed herein (e.g., fasudil, hydroxyfasudil, etc.) is administered orally or intravenously to the subject. In a certain embodiment, a ROCK inhibitor disclosed herein (e.g., fasudil, hydroxyfasudil, etc.) is formulated for oral delivery to the subject. In yet another embodiment, a ROCK inhibitor disclosed herein (e.g., fasudil, hydroxyfasudil, etc.) is administered at a dose of 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 400 mg, 500 mg, or a range that includes or is in between any two of the foregoing doses, including fractional increments thereof. In a further embodiment, the subject is administered daily from 1, 2, 3, 4, 5, 6 doses or a range of doses that includes any two of the foregoing values, of a ROCK inhibitor disclosed herein (e.g., fasudil, hydroxyfasudil, etc.). In yet a further embodiment, the subject has a condition or disorder that can be treated by increasing luminal distensibility of the organ. In a certain embodiment, the subject has kidney stone(s). In another embodiment, the subject is a mammalian subject. In yet another embodiment, the mammalian subject is a human subject.

In a particular embodiment, the disclosure also provides a method to treat or facilitate treatment of kidney stones in a subject in need thereof, comprising: administering a therapeutically effective dose of a ROCK inhibitor disclosed herein (e.g., fasudil, hydroxyfasudil, etc.) to the subject in need thereof, wherein administration of the therapeutically effective amounts of a ROCK inhibitor disclosed herein (e.g., fasudil, hydroxyfasudil, etc.) results in increased ureteral distensibility. In another embodiment, administration of the therapeutically effective dose of a ROCK inhibitor disclosed herein (e.g., fasudil, hydroxyfasudil, etc.) to the subject in need thereof, facilitates kidney stone removal by a medical procedure or by spontaneous passing. In yet another embodiment, the medical procedure is ureteroscopy. In a further embodiment, administration of the therapeutically effective dose of a ROCK inhibitor disclosed herein (e.g., fasudil, hydroxyfasudil, etc.) allows for usage of larger diameter medical instruments that were not safely usable prior to administration. In a certain embodiment, the fasudil derivative or analog is selected from ripasudil, H-1152, dimethylfasudil and Rho Kinase Inhibitor IV. In a particular embodiment, the therapeutically effective dose of a ROCK inhibitor disclosed herein (e.g., fasudil, hydroxyfasudil, etc.) is administered orally or intravenously to the subject. In another embodiment, a ROCK inhibitor disclosed herein (e.g., fasudil, hydroxyfasudil, etc.) is administered at a dose 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 400 mg, 500 mg, or a range that includes or is in between any two of the foregoing doses, including fractional increments thereof. In yet another embodiment, the subject is administered daily from 1, 2, 3, 4, 5, 6 doses or a range of doses that includes any two of the foregoing values, of a ROCK inhibitor disclosed herein (e.g., fasudil, hydroxyfasudil, etc.). In a further embodiment, the subject is a mammalian subject. In yet a further embodiment, the mammalian subject is a human subject.

For use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise a ROCK inhibitor disclosed herein (e.g., fasudil, hydroxyfasudil, etc.), optionally in a composition or in combination with another agent as disclosed herein. The container(s) optionally have a sterile access port (for example the container can be an intravenous solution bag or a vial having a stopper pierceable by a hypodermic injection needle). Such kits optionally comprise an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a ROCK inhibitor disclosed herein (e.g., fasudil, hydroxyfasudil, etc.). Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself, a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

The disclosure further provides that the methods and compositions described herein can be further defined by the following aspects (aspects 1 to 24):
1. A method to increase luminal distensibility of an organ in a subject, comprising:
administering a therapeutically effective dose of a Rho-associated protein kinase (ROCK) inhibitor to the subject,
wherein administration of the therapeutically effective dose of the ROCK inhibitor results in increased luminal distensibility in the organ of the subject.
2. The method of aspect 1, wherein the organ comprises a lumen surrounded by smooth muscle.
3. The method of aspect 1 or aspect 2, wherein the organ is selected from ureter(s), prostate, bladder, penis, gallbladder, gallbladder duct, and lung bronchiole.
4. The method of any one of aspects 1 to 3, wherein the organ is ureter(s) or prostate.
5. The method of any one of aspects 1 to 4, wherein the ROCK inhibitor is selected from belumosudil, fasudil, hydroxyfasudil, ripasudil, netarsudil, zelasudil, ROC-101, OXA-06, ROCK inhibitor-2, H-1152P, CPL409116, AT3148, RKI-1447, FSD-C10, verosudil, sovesudil, cotosudil, and a derivative or analog of any of the foregoing.
6. The method of any one of aspects 1 to 5, wherein the therapeutically effective dose of the ROCK inhibitor is administered orally or intravenously to the subject.
7. The method of any one of aspects 1 to 6, wherein the ROCK inhibitor is fasudil, hydroxyfasudil, or a fasudil derivative or analog.
8. The method of aspect 7, wherein the fasudil derivative or analog is selected from ripasudil, H-1152, dimethylfasudil and Rho Kinase Inhibitor IV.
9. The method of aspect 7 or aspect 8, wherein fasudil, hydroxyfasudil or a fasudil derivative or analog is administered at a dose of 10 mg to 100 mg.
10. The method of any one of aspects 7 to 9, wherein the subject is administered daily from 1 to 4 doses of fasudil, hydroxyfasudil and/or a fasudil derivative or analog.
11. The method of any one of aspects 1 to 10, wherein the subject has a condition or disorder that can be treated by increasing luminal distensibility of the organ.
12. The method of any one of aspects 1 to 11, wherein the subject has kidney stone(s).
13. The method of any one of aspects 1 to 12, wherein the subject is a human patient
14. A method to treat or facilitate treatment of kidney stone(s) in a subject in need thereof, comprising:
administering a therapeutically effective dose of a Rho-associated protein kinase (ROCK) inhibitor to the subject,
wherein administration of the therapeutically effective dose of the ROCK inhibitor results in increased ureteral distensibility.
15. The method of aspect 14, wherein administration of the therapeutically effective dose of a ROCK inhibitor to the subject in need thereof, facilitates kidney stone removal by a medical procedure or by spontaneous passing.
16. The method of aspect 15, wherein the medical procedure is ureteroscopy.
17. The method of any one of aspects 14 to 16, wherein administration of the therapeutically effective amounts of the ROCK inhibitor allows for usage of larger diameter medical instruments that were not safely usable prior to administration.
18. The method of any one of aspects 14 to 17, wherein the ROCK inhibitor is selected from belumosudil, fasudil, hydroxyfasudil, ripasudil, netarsudil, zelasudil, ROC-101, OXA-06, ROCK inhibitor-2, H-1152P, CPL409116, AT3148, RKI-1447, FSD-C10, verosudil, sovesudil, cotosudil, and a derivative or analog of any of the foregoing.
19. The method of any one of aspects 14 to 18, wherein the therapeutically effective dose of the ROCK inhibitor is administered orally or intravenously to the subject.
20. The method of any one of aspects 14 to 19, wherein the ROCK inhibitor is fasudil, hydroxyfasudil, or a fasudil derivative or analog.
21. The method of aspect 20, wherein the fasudil derivative or analog is selected from ripasudil, H-1152, dimethylfasudil and Rho Kinase Inhibitor IV.
22. The method of aspect 20 or aspect 21, wherein fasudil, hydroxyfasudil or a fasudil derivative or analog is administered at a dose of 10 mg to 100 mg.
23. The method of any one of aspects 20 to 22, wherein the subject is administered daily from 1 to 4 doses of fasudil, hydroxyfasudil and/or a fasudil derivative or analog.
24. The method of any one of aspects 14 to 23, wherein the subject is a human patient.

The following examples are intended to illustrate but not limit the disclosure. While they are typical of those that might be used, other procedures known to those skilled in the art may alternatively be used.

### EXAMPLES

The invention is illustrated in the following examples, which are provided by way of illustration and are not intended to be limiting.

Animal procurement and conditions. Female, non-genetically modified, Yorkshire pigs were procured from Premier BioSource at age 90-120 days and 35-45 kg at the time of procurement. All pigs received the following vaccines/parasite control medications prior to procurement: Iron Dextran, Lawsonia intracellularis vaccine, ivermectin, Mycoplasma hypopneumonia bacterin, G. parasuis+Streptococcus suis, Erysipelothrix rhusiopathiae vaccine, porcine circovirus vaccine (type 2), killed baculovirus vector, parvovirus Erysipelothrix rhusiopathiae/Leptospira bratislava/Canicola grippotyphosa/Hardjo-icterohaemorrhagiae pomona bacterin, and rotavirus type A, B, and C. The animals were acclimated at least 72-hours prior to entering the study and were housed singly to avoid aggression and facilitate accurate medication delivery. The pens were 5′×5′ and enrichment included two toys—Challenger ball and Kong Genius (Bio-Serv). The animals were provided with ad-lib water and were fed LabDiet 5084 at 2.5-3.5% of their body weight. Light cycle consisted of 11 hours on and 13 hours off. The animals were fasted the night prior to the initial sizing procedure and were given medication four hours prior to sedation on the day-seven sizing procedure.

Preparation of Smooth muscle relaxant (SMR) medications and control vehicle. The control group received one cup of sugar pudding daily (3.25 oz Snack Pack vanilla) without added medication. The following smooth muscle relaxant (SMR) medications were administered orally, either mixed into sugar pudding or placed inside a Fig Newton for animals resistant to pudding:
Fasudil (80 mg): Supplied as powder aliquoted into microcentrifuge tubes, reconstituted with 1 mL of sterile water, and mixed into pudding once daily.
Mirabegron (50 mg): Two crushed 25 mg tablets mixed into pudding and given once daily.
Rowatinex (two capsules): Gel capsules placed into pudding or a Fig Newton and administered twice daily. Each capsule contains the following terpenes: pinene (α+β) 31 mg, camphene 15 mg, cineol 3 mg, fenchone 4 mg, borneol 10 mg, anethol 4 mg, and olive oil 33 mg.
Silodosin (8 mg): Capsule opened, and contents mixed into pudding and administered once daily.
Tadalafil (20 mg): Two crushed 10 mg tablets mixed into pudding and given twice daily.
Verapamil (180 mg extended release): Crushed tablet mixed into pudding and given once daily.
   Note that the medication/vehicle contents were placed in a metal bowl and left in the feeder for the animal to consume. The handler remained in the room to ensure the animal consumed all of the contents.

Study design. The juvenile, female Yorkshire swine animal model was chosen for use in this experiment due to its histological, physiological, and anatomical similarities to the human ureter in addition to its generally similar metabolism of medication. To date, the average baseline diameter of a swine ureter and the endogenous factors influencing its size have not been well established. The average diameter of a swine ureter as well as endogenous factors that influence the diameter of a swine ureter are currently unknown and had to be determined and adjusted for. To define a physiological baseline, a pooled cohort of 136 female juvenile Yorkshire swine ureters were retrospectively reviewed. The swine ureters were sized utilizing urethral dilators at a 3.5 N threshold according to a previously established protocol. Given that urethral dilators only come in even sizes, the mean ureter size was rounded to the nearest even dilator size (10-24 Fr) and univariable and multivariable logistic regression analyses identified factors associated with ureteral size greater than the mean. These endogenous factors were then utilized as adjustments to better isolate effect of each medication on the ureter.

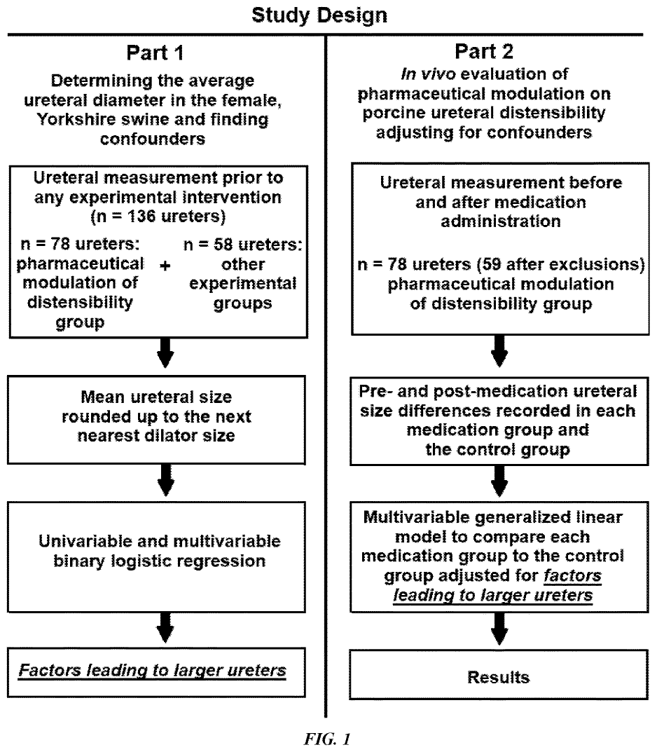

Once the endogenous variables associated with increased ureteral distensibility were established for the purpose of adjustment during analysis to isolate medication effects, a prospective, randomized trial was conducted to evaluate six oral smooth muscle SMRs versus placebo. Power was based on a study of female porcine ureter peristalsis modulation, and nine ureters, necessitating entering five animals, were required since this study utilized two ureters per animal. A multivariable generalized linear model was constructed to assess the medication's effect on increasing ureteral size compared to the control porcine ureters. The medication's impact on ureteral size was adjusted for age and the presence of bacteriuria at day zero or day seven. Bacteriuria was included in this model due to its importance in a clinical cohort and potential that its low representation (32% clinical versus 11% current study) might prevent capture as a logistic regression factor. The general experimental design is presented in FIG. 1.

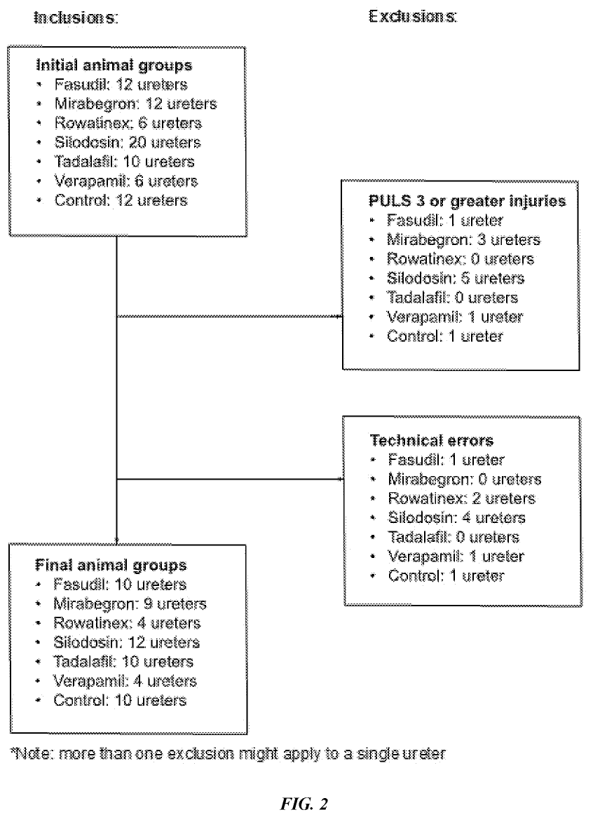

Ureteral sizing and medication delivery. All animals (n=35 (59 ureters) after exclusions) underwent initial ureteral sizing and were then randomized to receive fasudil, mirabegron, Rowatinex®, silodosin, tadalafil, verapamil, or a sugar pudding control (see Table 1), administered daily for one week. The animals were randomized using a random number generator to determine which drug each animal would receive, and the surgeon was blinded prior to and during all procedures. Ureteral sizing was accomplished by sequentially passing urethral dilators in 2 Fr increments using a UCI force sensor at a 3.5 Newtons (N) force limit. Bilateral ureteral sizing was done before and after a continuous seven-day administration of a selected oral medication. Ureters were excluded if they had evidence of a full thickness ureteral tear due to the initial sizing procedure (i.e., a post-ureteroscopic lesion scale (PULS) injury grade ≥3), or if a 10 Fr urethral dilator could not be passed with minimal force which was recorded as a “technical error” (e.g., see FIG. 2).

Animals were randomized to receive one of the following six SRMs or no medication (control group) in a cup of sugar pudding after the initial sizing procedure on day zero and continued to the morning of the second sizing procedure on day seven: fasudil, mirabegron, Rowatinex®, silodosin, tadalafil, or verapamil (see Table 1). Medication preparation details can be found below.

TABLE 1
Schedule of medications and vehicle/control.

| Medication | Dose | | Frequency | Form |
|---|---|---|---|---|
| Fasudil | 80 | mg | Daily | Powder |
| Mirabegron XR | 50 | mg | Daily | Crushed tablet |
| Rowatinex | 2 capsules | | Twice per day | Gel capsule |
| Silodosin | 8 | mg | Daily | Opened capsule |
| Tadalafil | 20 | mg | Daily | Crushed tablet |
| Verapamil XR | 180 | mg | Daily | Crushed tablet |

| | | | |
|---|---|---|---|
| Sugar pudding | 1 cup | Daily | Semi-solid |

Each medication was prepared and mixed into sugar pudding as described below. If an animal was resistant to the Rowatinex ® gel capsule, the medication was added to a Fig Newton (n = 1). Note that each Rowatinex ® capsule consists of the following terpenes: pinene [α + β] 31 mg, camphene 15 mg, cineol 3 mg, fenchone 4 mg, borneol 10 mg, anethol 4 mg, and olive oil 33 mg.

Age and weight measurement. The age and weight of the animals were recorded at day zero and day seven.

Ureteral sizing procedure on day zero and day seven. Two endourology fellows performed all the surgical procedures independently. Under general anesthesia with xylazine and isoflurane, each pig was intubated in a supine position; the abdomen and perineum were scrubbed with betadine and draped. Blood was drawn from the jugular vein at the start of each case for plasma drug level analysis. Flexible cystoscopy was performed, and a 0.035-inch Motion™ hybrid guidewire (Cook Medical, Bloomington, IN) was advanced into the renal pelvis under fluoroscopic guidance; this was accomplished for both ureters. Next, a 5 Fr open-ended ureteral catheter (Cook Medical, Bloomington, IN) was advanced over the guidewire until it reached the renal pelvis; this was completed on both sides under fluoroscopic guidance. Each guidewire was removed, and a retrograde pyelogram was performed bilaterally. A 0.035 inch Amplatz Super Stiff™ guidewire (Boston Scientific, Marlborough, MA) was passed via the 5 Fr open ended ureteral catheter, bilaterally. The 5 Fr catheters were removed.

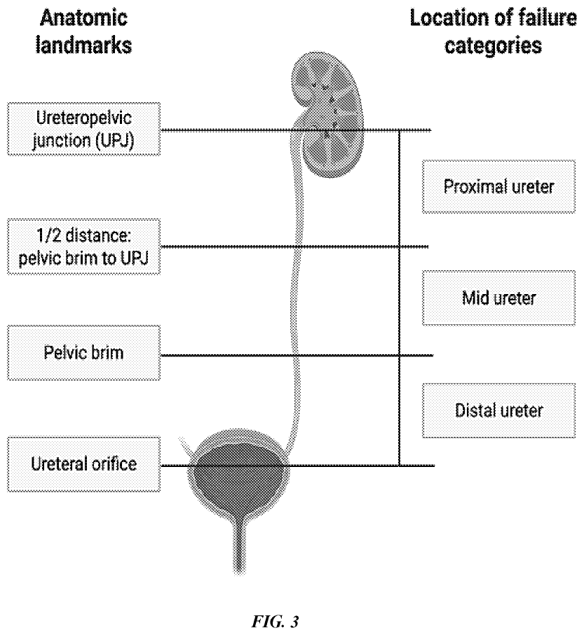

Polyethylene urethral dilators (Cook Medical, Bloomington, IN) were immersed in normal saline, coated with KY Jelly, and advanced sequentially in 2 Fr increments over the Super Stiff™ guidewire using the UCI-FS. If a urethral dilator advanced to the renal pelvis at ≤3.5 N, the next dilator in sequence was passed. If the force reached 3.5 N, the dilator's fluoroscopic location was recorded (i.e., distal, mid, or proximal ureter, e.g., see FIG. 3), and the dilator was removed. After sizing the ureter, an 8.5 Fr Flex XC ureteroscope (Karl Storz, Tuttlingen, Germany) was advanced over the guidewire until it reached the renal pelvis. The guidewire was removed. As the ureteroscope was withdrawn, the condition of the ureter was noted and a PULS grade was assigned to the proximal, middle, and distal ureter. The sizing was repeated for the contralateral ureter.

Post-operative medication administration. Following emergence from general anesthesia, a non-steroidal anti-inflammatory medication, flunixin meglumine, was given; this was repeated daily as needed to relieve any noted discomfort for up to three days. All animals received an antibiotic, enrofloxacin, at the time of the procedure and for three days postoperatively. Each animal was monitored for signs and symptoms of pain including behavioral changes (lethargy, aggression, unusual lying positions, and reduced food intake) and physical changes of unusual movement, labored breathing, vocalization, facial expressions, tremors, weight loss, or hematuria. No animals were found to be in discomfort or required euthanasia prior to the planned completion of the study. The selected oral SMR was then administered daily for seven days postoperatively. Dosage information is provided in Table 1, and SMR preparation details are provided above.

Ureteral sizing measurement on day seven. On day seven, the aforementioned ureteral sizing procedure was performed at least four hours after the pig received its final SRM dose. Following the final ureteral sizing procedure, the animal, while still under anesthesia, was chemically euthanized.

Ureters examined as independent units. Contralateral ureters in the same animal have variability at the baseline ureteral measurement. Thirty-two percent of swine were observed to have ≥3 USS points difference between their two ureters (e.g., see FIG. 4) in a pooled analysis of animals whose ureters were both measured prior to any experimentation (n=113) as opposed to single ureter measurements (n=136).

Data collection: ureteral size, demographics, and plasma drug levels. On days zero and seven, the largest urethral dilator that successfully reached the renal pelvis at <3.5 N of force was recorded for each ureter. If 3.5 N was reached before full advancement, the dilator size and its fluoroscopic position within the distal, mid, or proximal ureter was recorded. This secondary measurement, which took into account the distance within the ureter traversed by the final urethral dilator, was used to create a ureteral size scale (USS).

The calculation details are as follows: The size of the largest urethral dilator that successfully passed to the level of the renal pelvis under 3.5 N was recorded. Also recorded was the size of the urethral dilator and its ureteral position when 3.5 N was reached. This second measurement was reported as a ureteral size scale (USS) using EQ. 1.

USS=[(D_{f}−10)×3]+L_{f} (EQ. 1)

USS: ureteral size scale. D_{f}: urethral dilator size that failed to pass in Fr. L_{f}: location of urethral dilator failure, where proximal=3, middle=2, and distal=1.
The USS is calculated in two steps. First, each urethral dilator size from 10 Fr to the urethral dilator size that reached the 3.5 N failure point (D_{f}), is assigned three points [(D_{f}−10)×3]. Second, additional points are added for the ureteral location of dilator failure (L_{f}): proximal—three points, middle—two points, or distal—one point.

Ureteral size scale (USS) determination and example calculation. To quantify ureteral distensibility, a ureteral size scale (USS) was developed. Three points were awarded for each 1 Fr increase in dilator size successfully advanced to the ureteropelvic junction (UPJ), starting from a baseline of 10 Fr. An additional point was assigned based on the ureteral segment reached by the largest dilator at the 3.5 N force threshold: one point for distal, two points for mid, and three points for proximal ureteral advancement. For example, a 12 Fr dilator advanced to the mid ureter would yield a USS of 8 points-3 points each for 10 Fr and 11 Fr, plus 1 point for distal and 1 point for mid ureteral location.

Blood and urine collection. On days zero and seven, 3 mL of blood were collected in heparinized tubes (BD, NJ, USA); also, a catheterized urine specimen for urine culture was obtained. The collected blood samples were immediately centrifuged at 2,000×g for 15 minutes at 4° C., followed by transfer of the plasma into a sterile microcentrifuge tube for storage at −20° C. Plasma drug levels were analyzed using liquid chromatography-tandem mass spectrometry (LC-MSMS), as detailed below. Catheterized urine specimens were collected for culture on both the initiation and at the termination of the study in each pig.

Detection of plasma-drug levels using LC/MSMS. Analytical grade mirabegron, ranitidine, fasudil, hydroxyfasudil, tadalafil, sildenafil, verapamil, and metoprolol were obtained from Sigma-Aldrich (Millipore Sigma, MO, USA). Analytical grade mirabegron-d₅ was obtained from Sussex Research (Sussex Research, Ontario, CA). Analytical grade silodosin-d₄ was obtained from Cayman Chemicals (Cayman Chemicals, MI, USA). All other chemicals were obtained from Millipore Sigma, unless otherwise specified.

All quantitative analyses of drug levels were conducted using the Waters ACQUITY high-performance liquid chromatography (UPLC) triple quadrupole mass spectrometer (MS) instrument (Waters Corp., MA, USA). The chromatographic separation was performed on a Waters BEH C18 4.6×50 mm (1.7 μm particle size) column. Data was collected in positive ionization mode for all analytes with cone energy at 10 V and collision energy at 30 eV. Quantification was performed using multiple reaction monitoring (MRM) of the transitions m/z 390.1→268.1 and 475.1→100 for tadalafil and sildenafil (IS), 496.5→261.1 and 500.5→261.1 for silodosin and silodosin-d4 (IS), 455.2→165.1 and 268.1→116.1 verapamil and metoprolol (IS), and 397.3→146.0 and 402.4→384.1 mirabegron and miabegron-d5 (IS), 308.4→99.1 and 315.1→125 for hydroxyfasudil and ranitidine (IS), respectively.

Standard calibration curves were obtained using serial dilutions of each drug compound to give final concentrations of 1000, 333, 111, 37, 12, 4, 1.5, 0.5, 0.2, and 0.0 nM, with each dilution containing 100 nM of an internal standard specific to the drug compound. Quality control (QC) samples for each compound were prepared using pooled plasma from control pigs (day zero), with corresponding spiked drug compounds at 1000 nM, 100 nM, 1 nM, and 0 nM (matrix only), each containing a corresponding internal standard at a final concentration of 100 nM post-extraction.
Tadalafil: The extraction method was performed using a protocol derived from Ramakrishna et al., J Chromatogr B Analyt Technol Biomed Life Sci. 809(2):243-9 (2004). The internal standard, sildenafil, was spiked into 100 μL of plasma samples of animals from days 0 and 7. Then, 1 mL of acetonitrile solution was added to each sample and vortexed for 10 seconds. Each sample was then centrifuged at 13,000×g for 5 minutes. The supernatant was carefully collected, transferred, and evaporated in a chemical hood in a 40° C. water bath. The residue was then reconstituted in 500 μL of acetonitrile:water (50:50, v/v).
Fasudil: The extraction method was adapted from Chem et al., J Pharm Biomed Anal. 52(2):242-8 (2010). Its metabolite, hydroxyfasudil, was measured in the plasma samples for fasudil. The internal standard, ranitidine, was spiked into 200 μL of plasma samples of animals from days 0 and 7. Then, 500 μL of methanol was added to each sample, followed by vortexing. The tubes were then centrifuged at 11,000×g for 5 minutes. The Supernatant was carefully removed, collected, and evaporated in a chemical hood in a 40° C. water bath. The residue was reconstituted in 200 μL of methanol:water (50:50, v/v).
Silodosin: The extraction method was adapted from Shah et al., Biomed Chromatogr. 32(2) (2018). Silodosin-d4 (internal control) was spiked in aliquots of 300 μL of plasma samples of animals from days 0 and 7. Then, 100 μL of 0.1 M NaOH was added to each sample, followed by 30 seconds of vortexing. The analyte and internal standard were extracted by adding 2.5 mL of ethyl acetate-methyl tert-butyl ether (MTBE) (50:50, v/v). The solutions were incubated for 10 mins on a rotary mixer at 32×g. The samples were then centrifuged at 3200×g for 5 minutes. The organic layer of each sample was collected and evaporated in a chemical hood at a 40° C. water bath. The dried samples were reconstituted with 300 μL of 10 mM ammonium formate and methanol-acetonitrile (40:60, v/v) in a 20:80 (v/v) ratio solution.
Verapamil: The extraction method was adapted based on work by Borges et al., J Chromatogr B Analyt Technol Biomed Life Sci. 827(2):165-72 (2005). The internal standard, metoprolol, was spiked into 200 μL of plasma samples from days 0 and 7 of each animal. Verapamil and metoprolol were extracted with 4 mL of diethyl ether. Each sample was then vortexed for 60 seconds. The organic layer was collected and evaporated in the chemical hood at a 40° C. water bath. The dry residues were reconstituted using 200 μL water:methanol (30:70, v/v) solution.
Mirabegron: The extraction method was adapted from Teijlingen et al., J Chromatogr B Analyt Technol Biomed Life Sci. 887-888:102-11 (2012). In short, extraction of mirabegron began by aliquoting 500 μL of plasma from days 0 and 7 of each animal, followed by spiking mirabegron-d5 into each sample. Then, 400 μL of 20 mM ammonium acetate solution was added, and the samples were thoroughly mixed. Liquid-liquid extraction was performed by adding 3 mL of diethyl ether, shaken for 10 mins and then centrifuged for 5 mins at 2,500×g. The upper organic layer was collected and transferred to a new tube, evaporating to dryness in a chemical hood at a 40° C. water bath. The residues were reconstituted in 250 μL of 20 mM ammonium acetate:acetonitrile (70:30, v/v) solution.

Estimated Human Serum Plasma Levels. The mean time of dose to procedure (hr), mean porcine serum medication level (ng/mL), and the estimated human serum medication level (ng/mL) at the time of procedure were as follows: hydroxyfasudil—6.43, 9.260, 12 (fasting status unknown); mirabegron—4.87, 0.834, 30 (fasted); silodosin—5.23, 236, 51 (not fasted); tadalafil—5.25, 52, 450 (not fasted); verapamil—5.83, 27, 40 (fasting status unknown, 240 mg)).

Ureteral measurements outside of the protocol schedule. Two animals had their ureters measured on day eight rather than day seven. One control animal was measured on day eight (dilator size change for both ureters: greater than the mean, within one standard deviation; USS change: greater than the mean, one ureter within one standard deviation, one ureter within two standard deviations). One fasudil animal was sized on day eight and received one extra dose of the medication (dilator size change and USS change were greater than the mean, and both ureters were within one standard deviation for dilator size and USS).

Experimental animal characteristics. Pre-medication age, weight, and ureteral size of animals in each drug group as well the control groups are shown in Table 2 and the prevalence of bacteriuria pre- and post-medication administration in Table 3. There was no statistical difference in the size of the porcine ureter prior to medication administration between each experimental group and the control; however, some the Mirabegron, Silodosin, Tadalafil, and Verapamil groups were younger, and the Mirabegron group was lighter (see Table 2).

TABLE 2
Characteristics of experimental porcine ureter medication groups evaluated
at a 3.5N force threshold prior to medication administration.
A.

| | | | | | | Adjusted P value |
|---|---|---|---|---|---|---|
| Group | Parameter | Mean (95% CI) | SD | Median | IQR | vs. control* |
| Fasudil | Weight (Kg) | 41.35 (39.5-43.2) | 2.5 | 41.8 | 4.4 | 0.6886 |
| | Age (days) | 96.8 (92.2-101.4) | 6.4 | 98.0 | 14.0 | 0.0862 |
| | Ureter size (Fr) | 11.4 (10.2-12.6) | 1.6 | 11.0 | 2.5 | 0.6112 |
| Mirabegron | Weight (Kg) | 38.7 (37.8-39.7) | 1.2 | 39.5 | 2.2 | 0.0484 |
| | Age (days) | 84.8 (82.2-87.3) | 3.3 | 87.0 | 6.0 | <0.0001 |
| | Ureter size (Fr) | 12.7 (11.1-14.2) | 2.0 | 14.0 | 4.0 | 0.9807 |
| Rowatinex | Weight (Kg) | 42.4 (36.3-48.4) | 3.8 | 41.2 | 6.9 | 0.9933 |
| | Age (days) | 98.0 (81.0-115.0) | 10.7 | 96.5 | 19.5 | 0.6344 |
| | Ureter size (Fr) | 14.5 (11.4-17.6) | 1.9 | 15.0 | 3.5 | 0.9996 |
| Silodosin | Weight (Kg) | 40.3 (39.2-41.4) | 1.8 | 39.5 | 3.0 | 0.2514 |
| | Age (days) | 89.0 (86.3-91.5) | 4.1 | 88.5 | 3.8 | <0.0001 |
| | Ureter size (Fr) | 11.5 (10.9-12.1) | 0.9 | 12.0 | 2.5 | 0.6103 |
| Tadalafil | Weight (Kg) | 42.2 (39.2-45.2) | 4.2 | 42.0 | 3.5 | 0.9484 |
| | Age (days) | 92.6 (87.6-97.6) | 7.0 | 89.0 | 9.0 | <0.0001 |
| | Ureter size (Fr) | 12.2 (11.0-13.4) | 1.8 | 12.0 | 4.0 | 0.8975 |
| Verapamil | Weight (Kg) | 39.2 (38.8-39.7) | 0.3 | 39.2 | 0.5 | 0.2954 |
| | Age (days) | 83.5 (80.7-86.3) | 1.7 | 83.5 | 3.0 | <0.0001 |
| | Ureter size (Fr) | 11.5 (9.9-13.1) | 1.0 | 12.0 | 2.5 | 0.8588 |
| Control | Weight (Kg) | 43.6 (40.4-46.7) | 4.3 | 41.0 | 7.0 | n/a |
| | Age (days) | 101.1 (96.1-106.0) | 6.9 | 102.5 | 6.2 | n/a |
| | Ureter size (Fr) | 13.8 (12.1-15.5) | 2.4 | 14.0 | 4.0 | n/a |

*Dunett's multiple comparsion test was applied to adjust the P value

B.

| | | | Pre- | | Post- | | Anytime |
|---|---|---|---|---|---|---|---|
| | Urine | Pre- | medication | Post- | medication | | percent |
| | culture | medication | percent of | medication | percent of | Anytime | of total |
| Group | result | count | total (%) | count | total (%) | count | (%) |
| Fasudil | Negative | 10 | 100 | 8 | 80 | 8 | 80 |
| | culture | | | | | | |
| | Bacteriuria | 0 | 0 | 2 | 20 | 2 | 20 |
| | Total | 10 | 100 | 10 | 100 | 10 | 100 |
| | ureters | | | | | | |
| Mirabegron | Negative | 5 | 56 | 6 | 67 | 4 | 44 |
| | culture | | | | | | |
| | Bacteriuria | 4 | 44 | 3 | 33 | 5 | 56 |
| | Total | 9 | 100 | 9 | 100 | 9 | 100 |
| | ureters | | | | | | |
| Rowatinex | Negative | 3 | 75 | 3 | 75 | 3 | 75 |
| | culture | | | | | | |
| | Bacteriuria | 1 | 25 | 1 | 25 | 1 | 25 |
| | Total | 4 | 100 | 4 | 100 | 4 | 100 |
| | ureters | | | | | | |
| Silodosin | Negative | 3 | 25 | 8 | 67 | 1 | 8 |
| | culture | | | | | | |
| | Bacteriuria | 9 | 75 | 4 | 33 | 11 | 92 |
| | Total | 12 | 100 | 12 | 100 | 12 | 100 |
| | ureters | | | | | | |
| Tadalafil | Negative | 6 | 60 | 2 | 20 | 2 | 20 |
| | culture | | | | | | |
| | Bacteriuria | 4 | 40 | 8 | 80 | 8 | 80 |
| | Total | 10 | 100 | 10 | 100 | 10 | 100 |
| | ureters | | | | | | |
| Verapamil | Negative | 4 | 100 | 4 | 100 | 4 | 100 |
| | culture | | | | | | |
| | Bacteriuria | 0 | 0 | 0 | 0 | 0 | 0 |
| | Total | 4 | 100 | 4 | 100 | 4 | 100 |
| | ureters | | | | | | |
| Control | Negative | 10 | 100 | 8 | 80 | 8 | 80 |
| | culture | | | | | | |
| | Bacteriuria | 0 | 0 | 2 | 20 | 2 | 20 |
| | Total | 10 | 100 | 10 | 100 | 10 | 100 |
| | ureters | | | | | | |

TABLE 3
Bacteriuria prevalence in experimental animals
Bacteriuria was present pre- or post-medication in all animal groups except Verapamil.

| | | | Pre- | | Post- | | Anytime |
|---|---|---|---|---|---|---|---|
| | Urine | Pre- | medication | Post- | medication | | percent |
| | culture | medication | percent of | medication | percent of | Anytime | of total |
| Group | result | count | total (%) | count | total (%) | count | (%) |
| Fasudil | Negative | 10 | 100 | 8 | 80 | 8 | 80 |
| | culture | | | | | | |
| | Bacteriuria | 0 | 0 | 2 | 20 | 2 | 20 |
| | Total | 10 | 100 | 10 | 100 | 10 | 100 |
| | ureters | | | | | | |
| Mirabegron | Negative | 5 | 56 | 6 | 67 | 4 | 44 |
| | culture | | | | | | |
| | Bacteriuria | 4 | 44 | 3 | 33 | 5 | 56 |
| | Total | 9 | 100 | 9 | 100 | 9 | 100 |
| | ureters | | | | | | |
| Rowatinex | Negative | 3 | 75 | 3 | 75 | 3 | 75 |
| | culture | | | | | | |
| | Bacteriuria | 1 | 25 | 1 | 25 | 1 | 25 |
| | Total | 4 | 100 | 4 | 100 | 4 | 100 |
| | ureters | | | | | | |
| Silodosin | Negative | 3 | 25 | 8 | 67 | 1 | 8 |
| | culture | | | | | | |
| | Bacteriuria | 9 | 75 | 4 | 33 | 11 | 92 |
| | Total | 12 | 100 | 12 | 100 | 12 | 100 |
| | ureters | | | | | | |
| Tadalafil | Negative | 6 | 60 | 2 | 20 | 2 | 20 |
| | culture | | | | | | |
| | Bacteriuria | 4 | 40 | 8 | 80 | 8 | 80 |
| | Total | 10 | 100 | 10 | 100 | 10 | 100 |
| | ureters | | | | | | |
| Verapamil | Negative | 4 | 100 | 4 | 100 | 4 | 100 |
| | culture | | | | | | |
| | Bacteriuria | 0 | 0 | 0 | 0 | 0 | 0 |
| | Total | 4 | 100 | 4 | 100 | 4 | 100 |
| | ureters | | | | | | |
| Control | Negative | 10 | 100 | 8 | 80 | 8 | 80 |
| | culture | | | | | | |
| | Bacteriuria | 0 | 0 | 2 | 20 | 2 | 20 |
| | Total | 10 | 100 | 10 | 100 | 10 | 100 |
| | ureters | | | | | | |

Assumption verification. This study made assumptions that an initial ureteral measurement would not affect the repeated ureteral measurements in addition to considering each ureter as an independent unit. The initial assumption was further corroborated based on baseline and day seven ureteral size of the control animals; these values were compared with a Mann-Whitney U test to determine if the measurement methods alone caused a ureteral size change (French size and USS). To investigate whether ureters should be considered as independent or biologic replicates, the size of 113 pooled, juvenile, female, Yorkshire swine paired ureters that were measured prior to any experimental interventions from this and other studies were evaluated for variability through observation of the number and magnitude of size differences between the left and right ureter. In addition, normality testing was performed on the pooled animal group and experimental group to ensure that appropriate statistical methods were employed.

Statistics. Univariable and multivariable logistic regression analyses were used to identify factors associated with ureteral size greater than the average ureteral size. A multivariable generalized linear model assessed each medication's effect on ureteral size (French size and USS) compared to the control, adjusting for confounding factors associated with increased ureteral size.

Normality tests were performed on the data sets to ensure the appropriate statistical tests were conducted. Non-parametric data were compared with the Mann-Whitney U test and additional observational assessments were performed.

Statistics were performed with IBM SPSS Statistics for Windows, Version 29.0 (IBM Corporation, Armonk, NY) and GraphPad Prism, Version 11.0.0 (GraphPad Software, LLC, Boston, MA).

The average female Yorkshire swine ureteral size is 13 Fr at the 3.5 N force threshold. In a pooled cohort of 136 female juvenile Yorkshire swine ureters from prior studies requiring pre-intervention ureteral sizing, the mean ureteral diameter at a 3.5 N force threshold was 12.1 Fr (see Table 4). These animals had a mean age of 92.5 days and a mean weight of 42.7 kg. Bacteriuria was present in nine animals (see Table 4). The animal ureter size and age were normally distributed, and the average ureteral size was rounded to 12 Fr to serve as a threshold in subsequent analyses.

TABLE 4
Characteristics of porcine ureters evaluated
at a 3.5N force threshold.
A

| Parameter | Mean (95% Cl) | SD | Median | IQR |
|---|---|---|---|---|
| Weight (Kg) | 42.7 (41.3-44.1) | 8.1 | 41.2 | 5.1 |
| Age (days) | 92.5 (90.6-94.3) | 10.6 | 89.0 | 17.0 |
| Ureter size (Fr) | 12.1 (11.8-12.5) | 1.9 | 12.0 | 4.0 |

B

| Urine culture result | Count | Percent of total |
|---|---|---|
| Negative culture | 127 | 93% |
| Bacteriuria | 9 | 7% |
| Total ureters | 136 | 100% |

A) The mean size of the porcine ureter measured with the UCI-FS at a threshold of 3.5N was 12.1 Fr. The mean weight and age were 42.7 kg and 92.5 days, respectively.
B) A negative urine culture was observed in 90% of the animals.

Female Yorkshire swine age was correlated with ureteral size greater than 14 Fr. Univariable binary logistic regression analyses were conducted to assess the association of age, weight, and bacteriuria with ureteral sizes greater than 12 Fr. Age and bacteriuria were significant predictors. n multiple univariable simple binary logistic regression analyses evaluating age, weight, and bacteriuria for association with an average ureteral size larger than 14 Fr, age was found to be significant. In a multivariable binary regression model evaluating age and bacteriuria for correlation with an average ureteral size larger than 14 Fr, age was found to be significant (see Table 5). In the multivariable model, including age and bacteriuria, age and bacteriuria remained significantly associated with larger ureteral size (Age OR: 1.074, 95% CI: 1.025-1.124, P=0.003; Bacteriuria OR: 6.518, 95% CI: 1.348-31.519; see Table 5).

TABLE 5
Predictive factors of ureter size greater than 12 Fr.

| | | | | | | |
|---|---|---|---|---|---|---|
| Parameter | S.E. | Wald | df | P-value | OR | 95% CI |

A

| | | | | | | |
|---|---|---|---|---|---|---|
| Age (days) | 0.023 | 9.357 | 1 | 0.002 | 1.073 | 1.025-1.122 |
| Weight (kg) | 0.055 | 0.015 | 1 | 0.901 | 1.007 | 0.904-1.122 |
| Bacteriuria | 0.790 | 5.718 | 1 | 0.017 | 6.620 | 1.406-31.168 |

B

| | | | | | | |
|---|---|---|---|---|---|---|
| Age (days) | 0.071 | 9.089 | 1 | 0.003 | 1.074 | 1.025-1.124 |
| Bacteriuria | 1.875 | 5.435 | 1 | 0.020 | 6.518 | 1.348-31.519 |

A In multiple univariable simple binary logistic regression analyses evaluating age, weight, and bacteria for association with a ureteral size larger than 12 Fr, age and bacteriuria were found to be significant.
B In a multivariable binary logistic regression model evaluating age and bacteriuria for correlation with an average ureteral size larger than 12 Fr, age and bacteriuria were found to be significant.

In the present study, bacteriuria was significantly associated with increased ureteral distensibility (1.46 Fr or 0.49 mm, P=0.001), see Table 6.

TABLE 6
The effect of six oral medications on ureteral distensibility.

| | | | | |
|---|---|---|---|---|
| Difference (Fr) | S.E. | Wald Chi-Sq | df | P-value |

A

| Fasudil | 1.216 | 0.547 | 4.941 | 1 | 0.026 |
|---|---|---|---|---|---|
| Mirabegron | −1.029 | 0.711 | 2.093 | 1 | 0.148 |
| Rowatinex | −1.128 | 0.707 | 2.545 | 1 | 0.111 |
| Silodosin | −1.260 | 0.690 | 3.337 | 1 | 0.068 |
| Tadalafil | −0.025 | 0.642 | 0.002 | 1 | 0.969 |
| Verapamil | 0.057 | 0.732 | 0.006 | 1 | 0.002 |
| Control | 0ª | — | — | — | — |
| Age | −0.018 | 0.025 | 0.489 | 1 | 0.484 |
| Bacteriuria | 1.459 | 0.448 | 10.603 | 1 | 0.001 |
| | Difference (USS) | S.E. | Wald Chi-Sq | df | P-value |

B

| | | | | | |
|---|---|---|---|---|---|
| Fasudil | 3.685 | 1.680 | 4.811 | 1 | 0.028 |
| Mirabegron | −2.327 | 2.310 | 1.015 | 1 | 0.314 |
| Rowatinex | −3.334 | 2.203 | 2.290 | 1 | 0.130 |
| Silodosin | −0.689 | 1.763 | 0.153 | 1 | 0.696 |
| Tadalafil | 0.912 | 1.755 | 0.270 | 1 | 0.603 |
| Verapamil | 1.799 | 2.306 | 0.608 | 1 | 0.435 |
| Control | 0ª | — | — | — | — |
| Age | −0.025 | 0.032 | 0.582 | 1 | 0.446 |
| Bacteriuria | 1.950 | 1.208 | 2.604 | 1 | 0.107 |

^{a}set to zero because this parameter is redundant
A In a generalized linear model adjusted for age and bacteriuria that assessed six oral medications compared to control in terms of dilator size, fasudil, verapamil, and bacteriuria significantly increased ureter size.
B In a generalized linear model adjusted for age and bacteriuria that assessed six oral medications compared to control in terms of USS, only fasudil significantly increased ureter size, and Rowatinex trended toward a decrease in ureteral size.

Fasudil significantly increases ureteral size and USS score. Of the 39 animals (78 ureters), 35 animals (59 ureters) were evaluable after applying the aforementioned exclusion criteria (i.e. failure of a 10 Fr dilator to pass or presence of a ureteral tear from the dilation process). Of note, more than one exclusion, at times, applied to a single ureter (e.g., see FIG. 2). A generalized linear model was constructed to assess the changes in ureteral size for each of the six oral medications compared to the changes in ureteral size of the control ureters, adjusted for age and bacteriuria.

Fasudil was found to significantly increase the size of the urethral dilator passed by 1.216 Fr (0.41 mm, P=0.026) and to increase the USS by 3.685 points (P=0.016). Verapamil had a statistically significant increase in the size of the urethral dilator that could be safely passed albeit of less magnitude (0.057 Fr or 0.18 mm, P=0.028).

Serum levels of administered medications. Serum concentrations were detectable for five of the six medications; Rowatinex®, due to its multi-terpene composition, precluded accurate quantification. Of note, fasudil undergoes rapid hepatic oxidation to its active metabolite, hydroxyfasudil; as such, hydroxyfasudil was measured as an indicator of fasudil uptake.

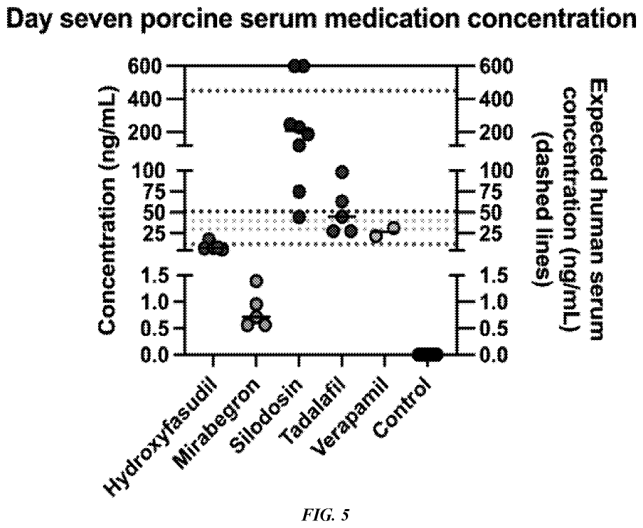

No drug serum levels were detectable on day zero. By day seven, all of the measurable five drugs were detectable in the serum of the treated animals. Mean serum concentrations on day seven were: hydroxyfasudil, 9.26 ng/mL (95% CI [3.39, 15.13]); mirabegron, 0.83 ng/mL (95% CI [0.40, 1.27]); silodosin, 263.00 ng/mL (95% CI [79.66, 446.35]); tadalafil, 52.26 ng/mL (95% CI [15.56, 88.93]); and verapamil, 26.65 ng/mL (95% CI [−34.15, 87.44]), see FIG. 5. Estimated human plasma levels of each of the five measurable drugs are detailed above and denoted as dashed lines corresponding to values on the right-side of the y-axis (e.g., see FIG. 5).

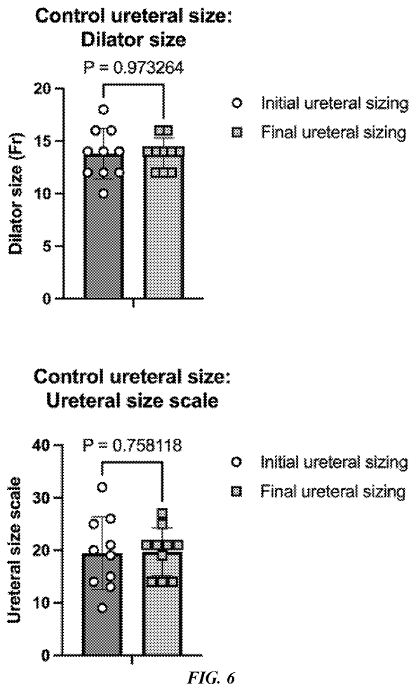

Serial ureteral measurements do not affect ureteral distensibility. The control group mean ureteral size at baseline was 13.8 Fr (SD 2.4), USS of 19.4 (SD 6.9) and the mean ureteral size at day-seven was 13.8 Fr (SD 1.5), USS of 19.7 (SD 4.5). There was no difference in mean ureteral size for diameter measurements (change of 0.0; P=0.9636) or USS measurements (change of 0.3; P=0.7506). The individual data points and summary data are provided in FIG. 6.

Conclusion. This study provides evidence that oral administration of fasudil and, to a lesser extent, verapamil resulted in a measurable increase in ureteral distensibility in a porcine model. Fasudil resulted in a statistically significant increase in both ureteral size, 1.216 Fr (0.41 mm), increase, and USS, 3.685 point, rise. While verapamil resulted in a statistically significant increase in ureteral size by 0.057 Fr (0.018 mm); the recorded increase in USS of 1.799 was not statistically significant. Among the six medications tested, fasudil was the only agent to produce a statistically significant and potentially clinically relevant increase in both measures of ureteral distensibility.

A number of embodiments have been described herein. Nevertheless, it will be understood that various modifications may be made without departing from the spirit and scope of this disclosure. Accordingly, other embodiments are within the scope of the following claims.

What is claimed is:

1. A method to increase luminal distensibility of an organ in a subject, comprising:
administering a therapeutically effective dose of a Rho-associated protein kinase (ROCK) inhibitor to the subject,
wherein administration of the therapeutically effective dose of the ROCK inhibitor results in increased luminal distensibility in the organ of the subject.

2. The method of claim 1, wherein the organ comprises a lumen surrounded by smooth muscle.

3. The method of claim 1, wherein the organ is selected from ureter(s), prostate, bladder, penis, gallbladder, gallbladder duct, and lung bronchiole.

4. The method of claim 1, wherein the organ is selected from ureter(s) and prostate.

5. The method of claim 1, wherein the ROCK inhibitor is selected from belumosudil, fasudil, hydroxyfasudil, ripasudil, netarsudil, zelasudil, ROC-101, OXA-06, ROCK inhibitor-2, H-1152P, CPL409116, AT3148, RKI-1447, FSD-C10, verosudil, sovesudil, cotosudil, and a derivative or analog of any of the foregoing.

6. The method of claim 1, wherein the ROCK inhibitor is fasudil, hydroxyfasudil, or a fasudil derivative or analog.

7. The method of claim 1, wherein the therapeutically effective dose of the ROCK inhibitor is administered orally or intravenously to the subject.

8. The method of claim 6, wherein fasudil, hydroxyfasudil or a fasudil derivative or analog is administered at a dose of 10 mg to 100 mg.

9. The method of claim 8, wherein the subject is administered daily from 1 to 4 doses of fasudil, hydroxyfasudil or a fasudil derivative or analog.

10. The method of claim 1, wherein the subject has a condition or disorder that can be treated by increasing luminal distensibility of the organ.

11. A method to treat or facilitate treatment of kidney stone(s) in a subject in need thereof, comprising:
administering a therapeutically effective dose of a Rho-associated protein kinase (ROCK) inhibitor to the subject,
wherein administration of the therapeutically effective dose of the ROCK inhibitor results in increased ureteral distensibility.

12. The method of claim 11, wherein administration of the therapeutically effective dose of the ROCK inhibitor to the subject in need thereof, facilitates kidney stone removal by a medical procedure or by spontaneous passing.

13. The method of claim 12, wherein administration of the therapeutically effective amounts of the ROCK inhibitor allows for usage of larger diameter medical instruments that were not safely usable prior to administration.

14. The method of claim 12, wherein the medical procedure is ureteroscopy.

15. The method of claim 11, wherein the ROCK inhibitor is selected from belumosudil, fasudil, hydroxyfasudil, ripasudil, netarsudil, zelasudil, ROC-101, OXA-06, ROCK inhibitor-2, H-1152P, CPL409116, AT3148, RKI-1447, FSD-C10, verosudil, sovesudil, cotosudil, and a derivative or analog of any of the foregoing.

16. The method of claim 11, wherein the ROCK inhibitor is fasudil, hydroxyfasudil, or a fasudil derivative or analog.

17. The method of claim 16, wherein the therapeutically effective dose of fasudil, hydroxyfasudil or a fasudil derivative or analog is administered orally or intravenously to the subject.

18. The method of claim 16, wherein fasudil, hydroxyfasudil or a fasudil derivative or analog is administered at a dose of 10 mg to 100 mg.

19. The method of claim 18, wherein the subject is administered daily from 1 to 4 doses of fasudil, hydroxyfasudil or a fasudil derivative or analog.

20. The method of claim 11, wherein the subject is a human patient.
